(19) United States
(12) Patent Application Publication
(10) Pub. No.: US 2021/0235994 A1
(43) Pub. Date: Aug. 5, 2021

(54) APPARATUS AND METHOD FOR AUTOMATIC IDENTIFICATION OF KOROTKOFF SOUNDS AND/OR BIOLOGICAL ACOUSTIC SIGNALS BY AN OPTICAL STETHOSCOPE

(71) Applicant: Pulse-Or Ltd, Rehovot (IL)

(72) Inventors: Evgeny Seider, Rehovot (IL); Yossi Kleinman, Rehovot (IL); Naum Chernoguz, Karmiel (IL); Alexander Finarov, Rehovot (IL); Ilya FINE, Rehovot (IL)

(21) Appl. No.: 17/173,934

(22) Filed: Feb. 11, 2021

Related U.S. Application Data
(63) Continuation of application No. PCT/IB2019/058237, filed on Sep. 27, 2019.
(60) Provisional application No. 62/737,191, filed on Sep. 27, 2018.

(51) Int. Cl. A61B 5/00; A61B 5/022

(52) U.S. Cl. CPC A61B 5/0082; A61B 5/02208; A61B 5/02233; A61B 5/7278; A61B 5/0066; A61B 5/726; A61B 5/7246; A61B 5/7203; A61B 5/02225; A61B 2562/0233; A61B 5/7282

(57) ABSTRACT

Methods and apparatus for optically detecting biologically-sourced acoustic signal(s) are disclosed herein. In some embodiments, K-sounds are detected and/or blood pressure is measured. Alternatively or additionally, an optical stethoscope (e.g. diffused-light interferometer optical stethoscope) is employed.

### RELATED APPLICATIONS

The present application gains priority from U.S. Provisional Patent Application 62/737191 filed on Sep. 27, 2018 and incorporated herein by reference in its entirety.

### BACKGROUND

The following pre-granted US patent publications provide potentially relevant background material, and are all incorporated by reference in their entirety: 20180279888 20180125377 20150366474 20140342332 20130289423 20120283584 20100106030 20100049093 20090227878 20080240345 20080089527 20080071179 20080033310 20070223652 20070016087 20060253040 20040147956 20030139674 20020143259.

The following issued US patents provide potentially relevant background material, and are all incorporated by reference in their entirety:

U.S. Pat. Nos. 9,974,449 9,934,701 8,483,399 7,634,049 7,512,211 7,485,131 6,805,671 6,705,998 6,605,103 6,511,435 6,231,523 5,967,993 5,840,036 5,651,369 5,649,535 5,560,365 5,447,162 5,406,954 5,406,953 5,388,585 5,316,005 5,218,967 5,203,341 5,135,003 5,103,830 5,099,851 5,031,630 4,974,597 4,972,841 4,971,064 4,967,756 4,961,429 4,938,227 4,889,132 4,867,171 4,840,181 4,768,519 4,677,983 4,635,645 4,607,641 4,592,366 4,592,365 4,549,549 4,534,361 4,501,281 4,476,876 4,473,080 4,459,991 4,432,373 4,429,700 4,396,018 4,356,827 4,337,778 4,326,536 4,320,767 4,313,445 4,262,674 4,252,127 4,248,242 4,202,347 4,181,122 4,116,230 4,112,929 4,105,020 4,068,654 4,058,117 4,026,277 4,005,701 3,930,494 4,396,018 4,058,117 4,592,366

FIG. 1A illustrates a prior art stethoscope. FIG. 1B illustrates a prior art blood-pressure measurement apparatus.

### SUMMARY

Apparatus for optically measuring blood pressure and/or detecting Korotkoff-sounds of an animal, the apparatus comprising: a. an inflatable cuff mechanically engageable to biological tissue of the animal; b. a diffused-light interferometer optical stethoscope comprising: i. a flexible and light-diffusing membrane; ii. a coherent-light source configured to emit light having a visible or NIR wavelength λ, the coherent-light source being aimed at a surface of the flexible membrane; and iii. a light-detector for receiving wavelength light λ that is emitted by the coherent-light source and reflected by the membrane, the diffused-light interferometer optical stethoscope being configured such that the wavelength light λ received by the light-detector is primarily light that is diffuse-reflected by the membrane; and c. Korotkoff-sound analysis circuitry for processing output of the light-detector that is generated when the flexible membrane is disposed over and/or mechanically engaged to and/or in contact with the cuff-engaged biological tissue, the Korotkoff-sound analysis-circuitry configured to detect Korotkoff-sounds from output of the light-detector.

In some embodiments, configured to optically measuring blood pressure and/or detecting Korotkoff-sounds of a human.

In some embodiments, the light-diffusing membrane is a multi-layer assembly comprising a light-diffusing film disposed over a membrane that is optionally light-diffusing.

In some embodiments, the light-diffusing membrane is substantially non-transparent to normally incident light of the wavelength λ so that optical density (OD) at wavelength λ is at least 2 or at least 3.

In some embodiments, the diffused-light interferometer optical stethoscope is configured such that at least 80% or at least 90% or at least 95% (by power) of wavelength light λ received by the light-detector is primarily light that is diffuse-reflected by the membrane.

In some embodiments, the coherent-light source is substantially normally aimed at a surface of the flexible membrane the coherent-light source being aimed at a surface of the flexible membrane, within a tolerance of at most 30° or within a tolerance of at most 20° or a tolerance of at most 15° or a tolerance of at most 10°.

In some embodiments, (i) the coherent-light source produces a light-spot on the flexible membrane; (ii) the flexible membrane is held substantially flat to define a horizon; and (iii) a line-segment connecting a center to the spot of light to a center of detector is at least one of: (i) between 45° and 65°; (ii) between 40° and 70°; (iii) between 35° and 75°) above the horizon defined by substantially flat membrane.

In some embodiments, the Korotkoff-sound analysis circuitry comprises at least one of hardware, a digital computer, analog circuitry, digital circuitry, software, firmware and computer-code.

In some embodiments, the Korotkoff-sound analysis circuitry detects the Korotkoff sounds based on analysis of temporal irregularities of output of the light-detector.

In some embodiments, further comprising blood-pressure detection circuitry for determining a systolic and/or diastolic blood pressure in accordance with a temporal correction between the Korotkoff-sound events and a pressure within the cuff.

In some embodiments, further comprising a pressure sensor for measuring a pressure within the inflatable cuff to generate an Oscillometric signal, and wherein the Korotkoff-sound analysis circuitry detects the Korotkoff events in accordance with a temporal correlation between (i) a pulsatile component of the Oscillometric signal and (ii) the output of the light-detector.

In some embodiments, the Korotkoff-sound analysis circuitry distinguishes between Korotkoff sounds and other biological acoustic signals.

In some embodiments, the Korotkoff-sound analysis circuitry detects the Korotkoff sounds by subjecting the optical signal to at least one of the following analysis techniques: entropy analysis, multiscale entropy analysis, fractal dimensions, multifractal analysis, wavelet analysis, Hurst exponential constants, pointwise Holder Exponent, and autocorrelation analysis.

Apparatus for optically detecting biological acoustic signals of an animal, the apparatus comprising:
a. an inflatable cuff mechanically engageable to biological tissue of the animal;
b. a diffused-light interferometer optical stethoscope comprising:
   i. a flexible and light-diffusing membrane;
   ii. a coherent-light source configured to emit light having a visible or NIR wavelength λ, the coherent-light source being aimed at a surface of the flexible membrane; and
   iii. a light-detector for receiving wavelength light λ that is emitted by the coherent-light source and reflected by the membrane,
the diffused-light interferometer optical stethoscope being configured such that the wavelength light λ received by the light-detector is primarily light that is diffuse-reflected by the membrane; and
c. biological-acoustic-signal analysis circuitry for processing output of the light-detector that is generated when the flexible membrane is disposed over and/or mechanically engaged to and/or in contact with the cuff-engaged biological tissue, the biological-acoustic-signal analysis configured to detect biological acoustic signals of the animal from output of the light-detector.

In some embodiments, biological-condition circuitry for detecting at least one of the following biological conditions according to output of the biological-acoustic-signal analysis circuitry: apnea events, abnormal heart murmurs Skin that appears blue, especially on your fingertips and lips; Swelling or sudden weight gain; Shortness of breath; Chronic cough; Enlarged liver; Enlarged neck veins; Poor appetite and failure to grow normally (in infants); Heavy sweating with minimal or no exertion; Chest pain; Dizziness; Fainting; Breath and Lung sounds; Small clicking, bubbling, or rattling sounds in the lungs. pneumonia, heart failure, and pleural effusion; Increased thickness of the chest wall; Over-inflation of a part of the lungs (e.g due to emphysema); reduced airflow to part of the lungs; Abdominal sounds made by the movement of the intestines; Gas; Nausea; Presence or absence of bowel movements; Vomiting; Audible vascular sounds called bruits that are caused by turbulent flow in large arteries; and Aneurisma.

In some embodiments, the detected biological acoustic signals is selected from the group consisting of: (I) Korotkoff-sounds; (ii) a pulsatile acoustic signals; (iii) breathing or a pulmonary acoustic signal; (iv) a digestive or bowel acoustic signal; (v) an acoustic signal produced by a fetus within the animal; and (vi) sounds made by the heart, lungs, intestines, blood vessels vibration and/or blood flow.

In some embodiments, the light-diffusing membrane is a multi-layer assembly comprising a light-diffusing film disposed over a membrane that is optionally light-diffusing.

In some embodiments, the light-diffusing membrane is substantially non-transparent to normally incident light of the wavelength λ so that optical density (OD) at wavelength λ is at least 2 or at least 3.

In some embodiments, the diffused-light interferometer optical stethoscope is configured such that at least 80% or at least 90% or at least 95% (by power) of wavelength light λ received by the light-detector is primarily light that is diffuse-reflected by the membrane.

In some embodiments, the coherent-light source is substantially normally aimed at a surface of the flexible membrane the coherent-light source being aimed at a surface of the flexible membrane, within a tolerance of at most 30° or within a tolerance of at most 20° or a tolerance of at most 15° or a tolerance of at most 10°.

In some embodiments, the (i) the coherent-light source produces a light-spot on the flexible membrane; (ii) the flexible membrane is held substantially flat to define a horizon; and (iii) a line-segment connecting a center to the spot of light to a center of detector is at least one of: (i) between 45° and 65°; (ii) between 40° and 70°; (iii) between 35° and 75°) above the horizon defined by substantially flat membrane.

In some embodiments, the biological-acoustic-signal analysis circuitry comprises at least one of hardware, a digital computer, analog circuitry, digital circuitry, software, firmware and computer-code.

In some embodiments, the biological-acoustic-signal analysis circuitry detects the biological acoustic signal based on analysis of temporal irregularities of output of the light-detector.

In some embodiments, the biological-acoustic-signal analysis circuitry detects the Korotkoff sounds by subjecting the optical signal to at least one of the following analysis techniques: entropy analysis, multiscale entropy analysis, fractal dimensions, multifractal analysis, wavelet analysis, Hurst exponential constants, pointwise Holder Exponent, and autocorrelation analysis.

A method for optically measuring blood pressure and/or detecting Korotkoff-sounds of an animal, the apparatus comprising:
a. engaging an inflatable cuff to biological tissue of the animal;
b. providing a diffused-light interferometer optical stethoscope comprising:
   i. a flexible and light-diffusing membrane;
   ii. a coherent-light source configured to emit light having a visible or NIR wavelength λ, the coherent-light source being aimed at a surface of the flexible membrane; and
   iii. a light-detector for receiving wavelength light λ that is emitted by the coherent-light source and reflected by the membrane, the diffused-light interferometer optical stethoscope being configured such that the wavelength light λ received by the light-detector is primarily light that is diffuse-reflected by the membrane;
c. mechanically coupling the flexible membrane of the optical stethoscope to the biological tissue of the animal so that mechanical vibrations of a biological acoustic signal are conveyed from the biological tissue to the flexible membrane; and
d. electronically processing output of the light-detector that is generated when the flexible membrane is disposed over and/or mechanically engaged to and/or in contact with the cuff-engaged biological tissue so as to electronically detect Korotkoff-sounds from output of the light-detector.

A method for optically detecting biological acoustic signals of an animal, the apparatus comprising:
a. providing a diffused-light interferometer optical stethoscope comprising:
   i. a flexible and light-diffusing membrane;
   ii. a coherent-light source configured to emit light having a visible or NIR wavelength λ, the coherent-light source being aimed at a surface of the flexible membrane; and
   iii. a light-detector for receiving wavelength light λ that is emitted by the coherent-light source and reflected by the membrane,
the diffused-light interferometer optical stethoscope being configured such that the wavelength light λ received by the light-detector is primarily light that is diffuse-reflected by the membrane;
b. mechanically coupling the flexible membrane of the optical stethoscope to the biological tissue of the animal so that mechanical vibrations of a biological acoustic signal are conveyed from the biological tissue to the flexible membrane; and and
c. electronically processing output of the light-detector that is generated when the flexible membrane is disposed over and/or mechanically engaged to and/or in contact with the cuff-engaged biological tissue so as to electronically detect the biological acoustic signals from output of the light-detector.

### BRIEF DESCRIPTION OF THE DRAWINGS

Some embodiments of the printing system are described herein with reference to the accompanying drawings. The description, together with the figures, makes apparent to a person having ordinary skill in the art how the teachings of the disclosure may be practiced, by way of non-limiting examples. The figures are for the purpose of illustrative discussion and no attempt is made to show structural details of an embodiment in more detail than is necessary for a fundamental understanding of the disclosure. For the sake of clarity and simplicity, some objects depicted in the figures are not to scale.

FIGS. 1A-1C describe prior art.

FIGS. 2-23 describe embodiments of the invention.

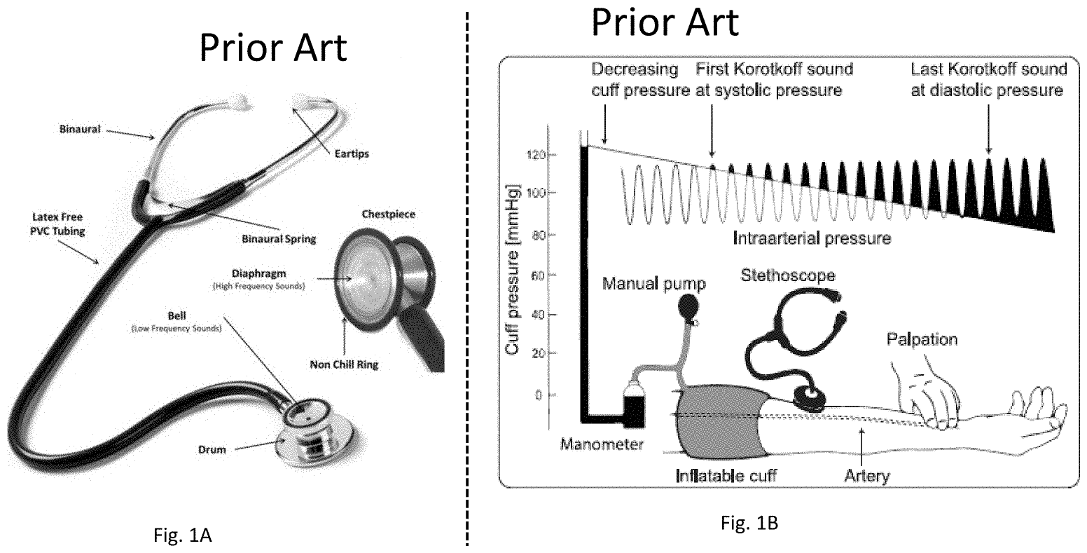

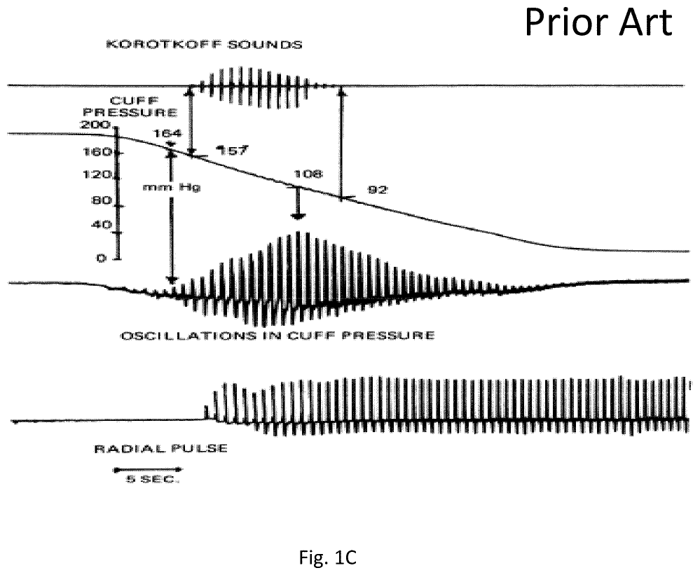

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

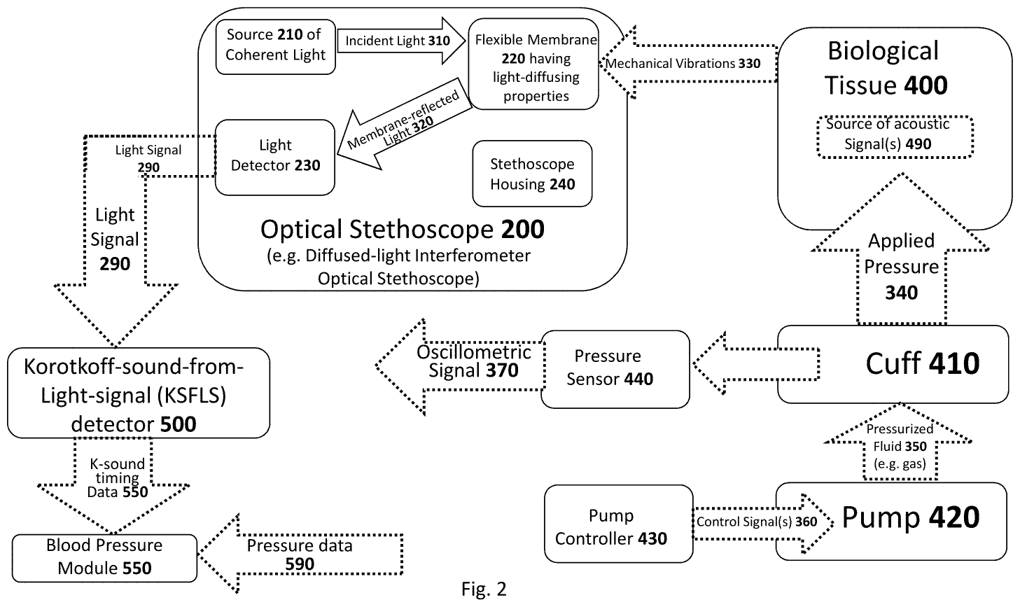

FIG. 2 is a block diagram of apparatus for optically detecting Korotkoff sounds—e.g. for the purpose of optically measuring blood pressure and/or arterial wall elasticity).

The apparatus includes: (i) inflatable cuff 410 for applying pressure 340 to biological tissue 400 (e.g. the wrist or forearm or finger); (ii) a pump 420 for inflating cuff 410; (iii) an optical stethoscope 200 for optically detecting acoustic signals(s) 490 of biological tissue 400; and (v) a Korotkoff-sound-from-light-signal (KSFLS) detector 500 for detecting Korotkoff sounds by analyzing output of optical stethoscope 200 of an element (e.g. light detector 230) thereof.

The ‘detecting’ of the Korotkoff sounds may include any one of (i) identifying a presence of Korotkoff sounds; (ii) identifying a time when Korotkoff sound(s) commence (e.g. to measure systolic blood pressure) or conclude (e.g. to measure diastolic blood pressure); (iii) characterizing or classifying a type of a detected Korotkoff sound (e.g. to distinguish between a Korotkoff of a more fit and a less fit individual); and (iv) computing one or more features of a Korotkoff sound.

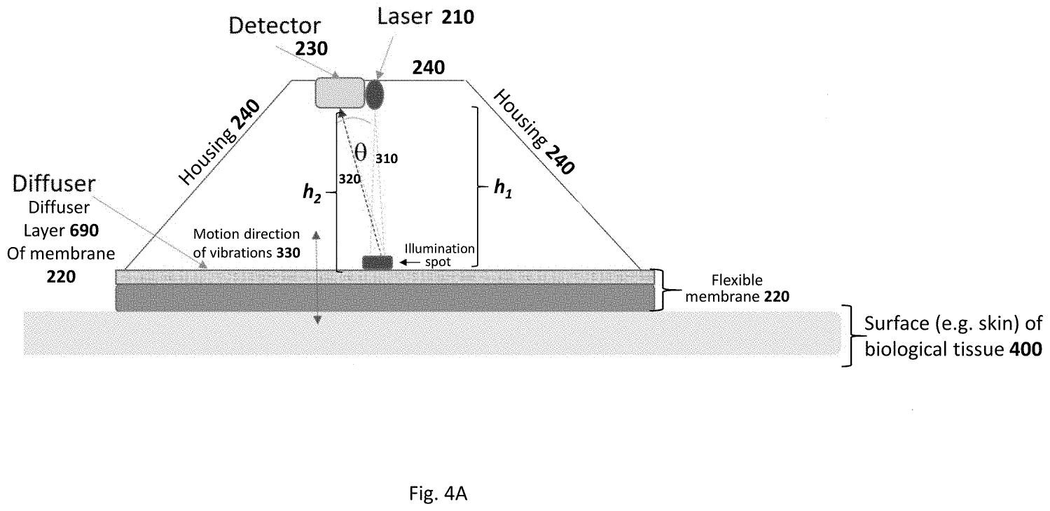

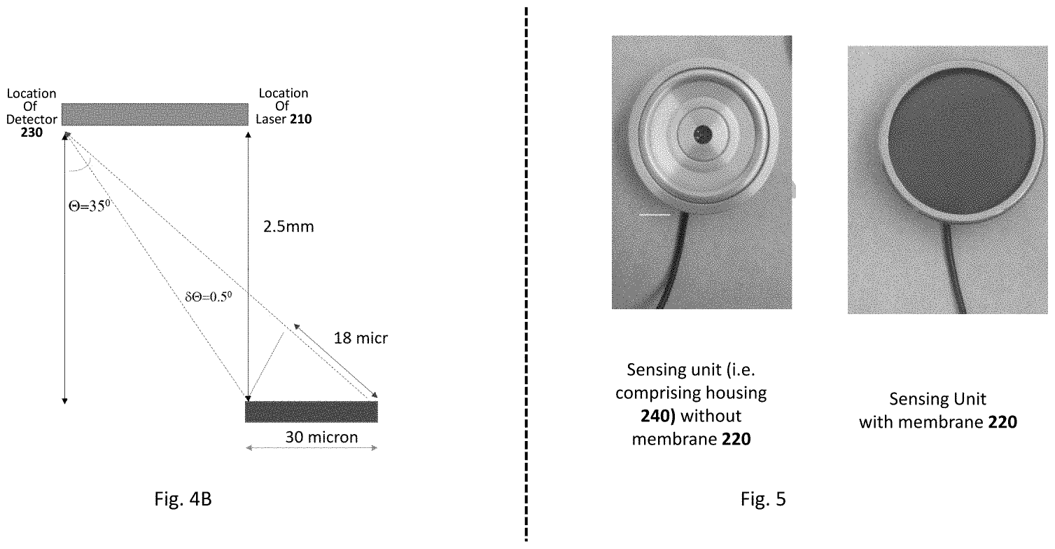

The optical stethoscope 200 or elements thereof are shown in FIGS. 4A-4B, 5, and 6A-6B. As shown in FIG. 4A: (i) source 210 of coherent light (e.g. laser) is disposed above (e.g. held above) an upper surface of membrane 220—e.g. at a height H1; and (ii) light detector 230 is disposed above (e.g. held above) an upper surface of membrane 220—e.g. at a height H2. Although FIG. 4A shows that H1=H2 this is not a requirement. In the example of FIG. 4A, a length of the shortest optical path from source 210 to detector 230 is H1+H2, where the total optical path has two parts: (i) a first part from source 210 to membrane 220 and (ii) a second part from membrane 220 back to detector 230.

As noted above, light source 210 emits coherent light—i.e. of a wavelength λ. λ is typically in the visible or infrared (e.g. NIR) range. For example, λ is on the same order of magnitude as an amplitude of mechanical vibrations 330 of membrane 220.

Although FIG. 4A shows a single source 210 and a single detector 230, this is not a limitation—any number of source(s) 210 and detector(s) 230 may be employed. In one example, multiple detectors receive membrane-reflected light 320 derived from a single light source 210. In embodiments of the invention, the optical stethoscope 200 works as follows: a flexible membrane 220 is mechanically coupled (e.g. disposed over and/or placed on—for example, in direct contact with the skin) to biological tissue 400. Acoustical signals (e.g. the noise of blood flow or pulse, Korotkoff sounds, breathing, heart sounds, aortic aneurism, abdominal sounds, bowel sounds, fetal sounds) generated by the subject propagate within the biological tissue.

Because of the mechanical coupling between (i) the flexible membrane 220 of optical stethoscope 200 and (ii) the biological tissue 400, acoustical signals from biological tissue drive mechanical vibrations 330 of the flexible membrane—for example, vibrations in a direction that is normal to the membrane and/or normal to the surface of the biological tissue. For example, an amplitude of these vibrations is on the order of magnitude of 0.1 μM-1M. Due to these vibrations, a length of the optical path from source 210 to detector 230 fluctuates in time—e.g. both an extent of light interference and a type (i.e. constructive vs. destructive) of light interference fluctuations in time.

Thus, optical stethoscope may be said to be an “interferometer.” The temporal fluctuations of membrane-reflected light 320 as received by detector 230 are driven by temporal changes in an extent of interference of light along the optical path from source 210 to detector 230.

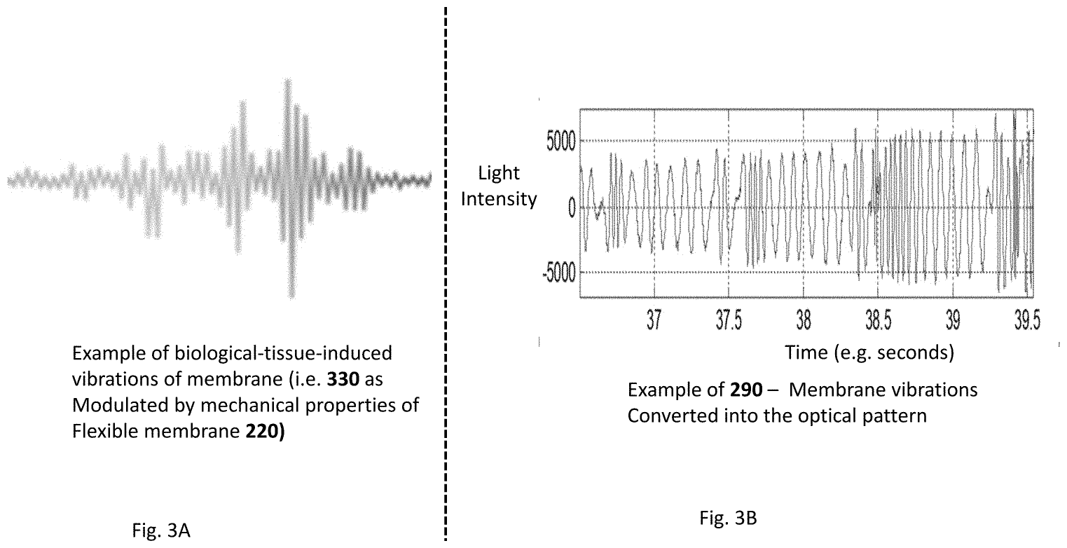

FIG. 3, which illustrates both (i) biological-tissue-induced vibrations 330 of membrane (i.e. acoustical signal within the biological tissue induce the vibrations); and (ii) these vibrations as “converted” into temporal fluctuations in intensity of light received by detector 230. It is believed that the vibrations of membrane 220 describe the actual acoustical signal within biological tissue 400, as modulated by mechanical properties of flexible membrane 220.

Light diffuser properties—It is known that there are two types of reflections: (i) specular reflections and (ii) diffuse reflections. In embodiments of the invention, membrane 220 is a light diffuser, or is associated with a light diffusive film or coating, or may be said to have light diffusive properties. Not wishing to be bound by theory, it is believed that the light-diffusive properties allow for “sampling” and an “averaging” of reflections over a larger horizontal area of membrane 220, which may provide for a more stable measurement that does not depend on reflections from a very ‘localized’ point on membrane 220. Furthermore, the diffusive reflections may also facilitate the use of multiple detectors 230.

The inventor is aware that light-diffusive properties may distort the signal—i.e. the light signal 290 emitted by detector 230 is distorted (i.e. due to the light-diffusive properties) with reflect to a signal of the mechanical vibrations 330. In this sense, stethoscope 200, in some embodiment and for certain sounds (e.g. K-sounds or other sounds having a frequency below hundreds of hertz) may be said to not be a ‘good’ microphone, due to these distortions.

In the non-limiting implementation of FIG. 4A, membrane 220 has multiple layers—e.g. a main layer and a diffusive film above the main layer. In other membrane 220 has a single layer. Even if the diffusive film is a ‘separate layer’ the light may be said to be diffuse-reflected by membrane 220.

The aforementioned diffuser properties and/or substantial non-transparency properties of membrane 220 at wavelength λ may contribute to a situation where λ wavelength light received by the light-detector 230 is primarily light that is diffuse-reflected by the membrane 220. This is in contrast to light which would traverse an entire thickness of membrane 220, exit from membrane 220 and then would be reflected by the biological tissue 400 (e.g. skin).

In embodiments of the invention, a thickness of flexible membrane 220 is at least 0.05 microns or least 0.1 microns. Alternatively or additionally, a thickness of flexible membrane 220 is at most 0.5 microns or most 0.4 microns or at most 0.3 microns.

In some embodiments, a flexible membrane 220 is between 0.3 and 0.5 microns.

In different embodiments of the invention, flexible membrane 220 or a later thereof is made of metal (e.g. steel or aluminum) or plastic—e.g. having optical properties to sufficiently reflect light of wavelength λ so that λ wavelength light received by the light-detector 230 is primarily light that is diffuse-reflected by the membrane 220.

Another element of optical stethoscope 200 is stethoscope housing 240, which is shown schematically in FIG. 4A-4B, and in the photograph (i.e. for one particular implementation) of

FIG. 5. One potential function of the housing 240 is to maintain positions of coherent-light source 210 and detector 230—e.g. constant relative to each other and/or relative to membrane 220 (e.g. when not vibrating).

In some embodiments, a value of H1 is at least 1 mm or at least 1.5 mm or at least 2 mm Alternatively or additionally, the value of H1 is at most 5 mm or at most 4 mm or at most 3 mm.

In some embodiments, a value of H2 is at least 1 mm or at least 1.5 mm or at least 2 mm Alternatively or additionally, the value of H2 is at most 5 mm or at most 4 mm or at most 3 mm.

FIG. 4B shows a very specific geometry—note that theta is 35° in this example. Not wishing to be bound by theory,

In some embodiments, (i) membrane 220 is held substantially flat (e.g. by housing 240) to define a horizon; (ii) source 210 illuminates membrane 220 with a light-beam that produces a spot of light on membrane 220; and (ii) a line-segment connecting a center to the spot of light to a center of detector 230 is 55° or about 55° (e.g. between 45° and 65°; e.g. between 40° and 70°; e.g. between 35° and 75°) above the horizon defined by substantially flat membrane 220). For example, the incident beam of light is substantially perpendicular to the horizon/plane of membrane 220—e.g. within a tolerance of at most 35° or at most 25° or at most 20° or at most 15° or at most 10° or at most 5°. For example, experimental evident appears to indicate that this confirmation may be optimal for signal-noise ratio where ‘signal’ is the light received by detector indicative of the Korotkoff sounds.

Another potential function of stethoscope housing 240 is to maintain membrane 220 flat. For example, even if housing 240 does not apply an active tensioning/stretching force to membrane 220, a presence of housing 240 may maintain membrane 220 flat—i.e. if one tries to deform or bend membrane 220 housing 240 would apply a counter-force to maintain membrane 220 flat. See, for example, FIG. 5.

In some embodiments, a spot-size of the spot produced by light source 210 is at most 100 μM or at most 75 μM or at most 50 μM or at most 30 μM.

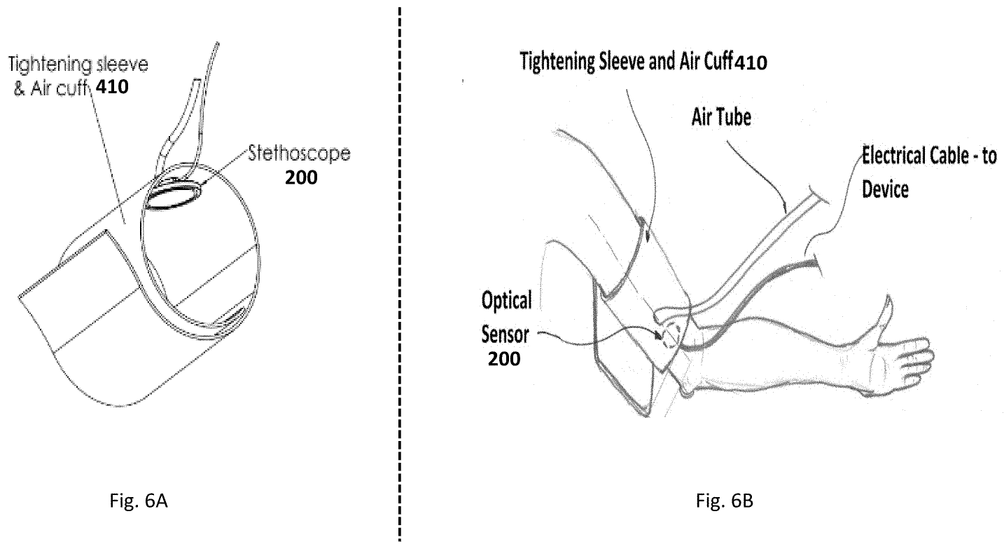

FIGS. 6A-6B show examples of K-sound detection apparatus including cuff 410 which applies pressure 340 to biological tissue 400 (e.g. arm or forearm or write or finger). As shown in FIG. 2, pump 420 may force pressurized fluid 350 into cuff—e.g. according to control signal(s) 360 produced by pump controller 430. Pressure sensor 440 may measure pressure within cuff 410 to produce Oscillometric signal 370.

Light detector 230 produces a light signal 290, whose content is analyzed, for example, by Korotkoff-sound-from-light-signal (KSFLS) detector 500. The purpose of KSFLS detector 500 is to ‘detect’ Korotkoff sounds from the output of light detector 230.

The detection of the Korotkoff sounds may be useful in and of itself. In some embodiments, the detection of Korotkoff is used (e.g. by blood pressure module) to measure systolic and/or diastolic blood pressure, e.g. by correlating a timing of the Korotkoff sounds with data describing the pressure within cuff 410 as a function of time. For example, the data describing the pressure within cuff 410 as a function of time may be provided by Oscillometric signal 370 and/or control signal(s) 360.

Alternatively or additionally, the form-factor of the Korotkoff sounds may be analyzed—e.g. for the purpose of computing arterial wall elasticity of the subject (e.g. of biological tissue 400).

For the present disclosure, ‘module’ and/or ‘electrical circuitry’ or ‘electronic circuitry’ (or any other ‘circuitry’ such as ‘blood pressure circuitry’ or ‘control circuitry’ or ‘pump control circuitry’) and/or element and/or unit and/or controller and/or module and/or sensor (e.g. 500 or 550 or 440 or 430 or 230) may include any combination of analog and/or digital circuitry and/or software/computer readable code module and/or firmware and/or hardware element(s) including but not limited to a digital computer, CPU, volatile or non-volatile memory, field programmable logic array (FPLA) element(s), hard-wired logic element(s), field programmable gate array (FPGA) element(s), and application-specific integrated circuit (ASIC) element(s). Any instruction set architecture may be used including but not limited to reduced instruction set computer (RISC) architecture and/or complex instruction set computer (CISC) architecture.

In different embodiments, any computation or analysis procedure may be performed using any combination of analog and/or digital circuitry and/or software/computer readable code module and/or firmware and/or hardware element(s) including but not limited to a digital computer, CPU, volatile or non-volatile memory, field programmable logic array (FPLA) element(s), hard-wired logic element(s), field programmable gate array (FPGA) element(s), and application-specific integrated circuit (ASIC) element(s). Any instruction set architecture may be used including but not limited to reduced instruction set computer (RISC) architecture and/or complex instruction set computer (CISC) architecture.

Detection of Korotkoff Sounds (K Sounds)

In some embodiments, the detection of the Korotkoff sounds (e.g. from light signal 290 or a derivative thereof) is based at least in part on optical signal pattern recognition and analysis of irregularities of the time dependent measured optical signal 290—e.g. during inflation or deflation of cuff 410.

Alternatively or additionally, the detection or identification of the Korotkoff sounds is based at least in part on spectrum analysis of the optical signal.

Alternatively or additionally, the detection or identification of the Korotkoff sounds is based at least in part on analysis of irregularities of the optical signal (e.g. 290) fluctuation.

Alternatively or additionally, the detection or identification of the Korotkoff sounds is based on the analysis of the correlation of the irregularities of the optical signal 290 fluctuation with the oscillometric pulse wave (e.g. oscillometric signal 370 or a component thereof—e.g. pulsatile and/or DC component).

In some embodiments, it possible to compute irregularities using a sliding window technique. In different embodiments, one or more of the following is computed (e.g. over the sliding window) to detect K-sounds and/or irregularities: entropy; entropy multiscale entropy, fractal dimensions, multifractal analysis, wavelet analysis, Hurst exponential constants, pointwise Holder Exponent, autocorrelation analysis.

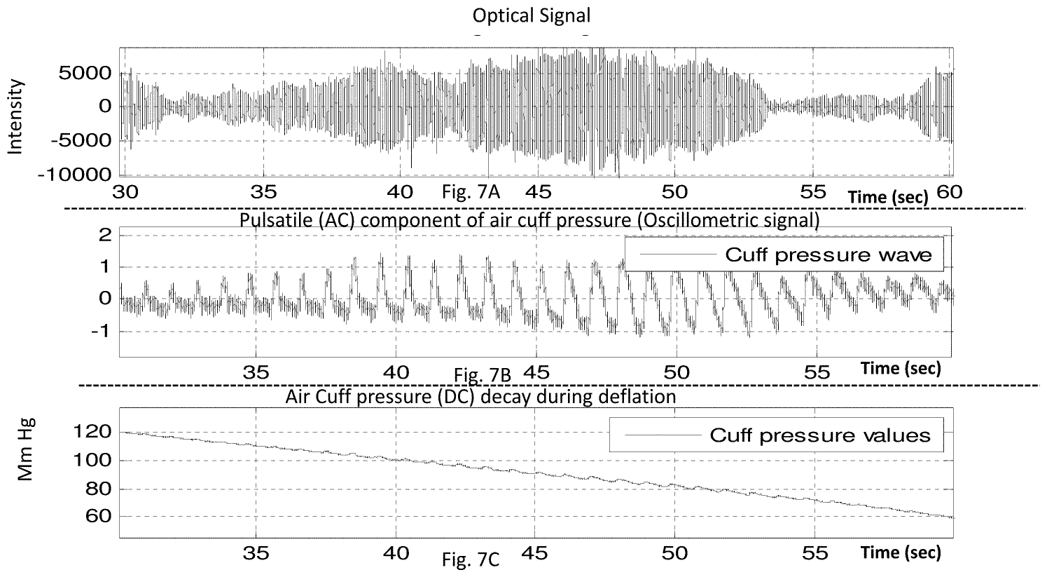

FIG. 7A illustrates an example light signal 290 produced by detector 230. FIG. 7B illustrates AC/pulsatile component of an example Oscillometric signal 370. FIG. 7C illustrates the DC component (e.g. pressure ramp-down) of example Oscillometric signal 370.

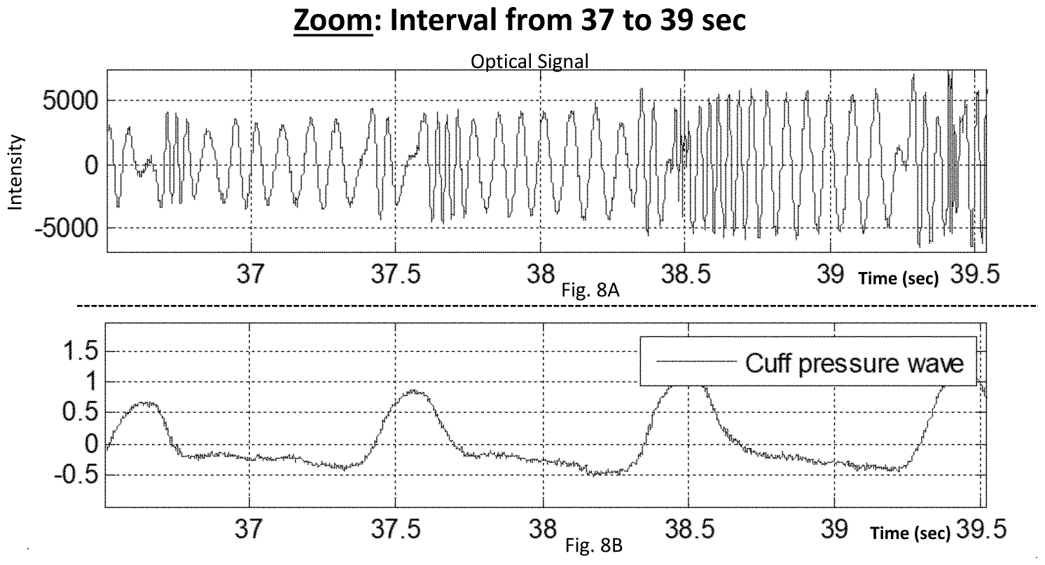

FIG. 8A-8B show the example light signal 290 and the AC/pulsatile component of an example Oscillometric signal 370 at a time of an appearance of the Korotkoff sound. By correlating between optical signal 290 and AC/pulsatile component of an Oscillometric signal 370 it is possible to more accurately detect Korotkoff sounds.

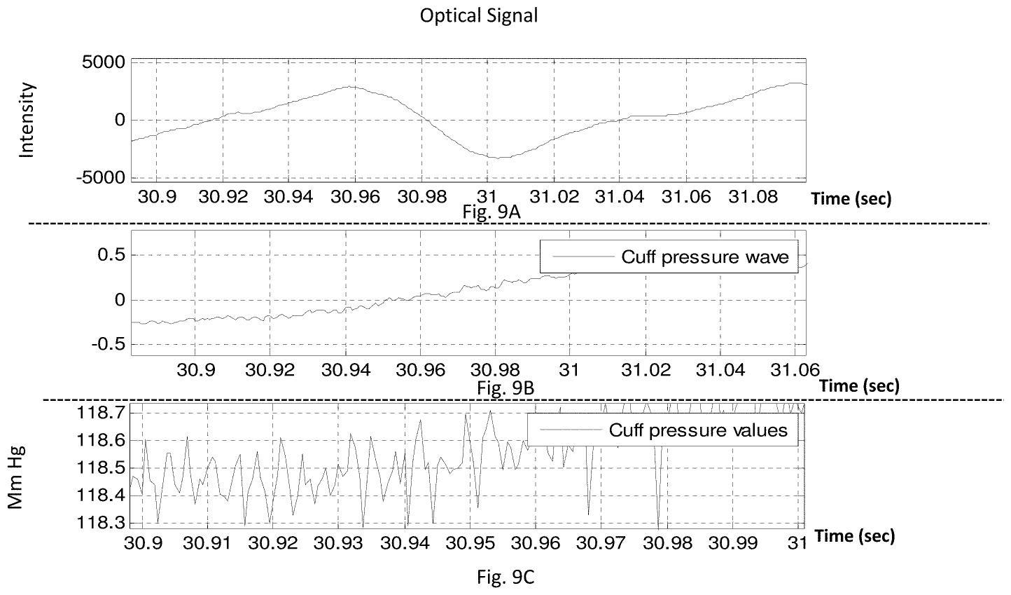

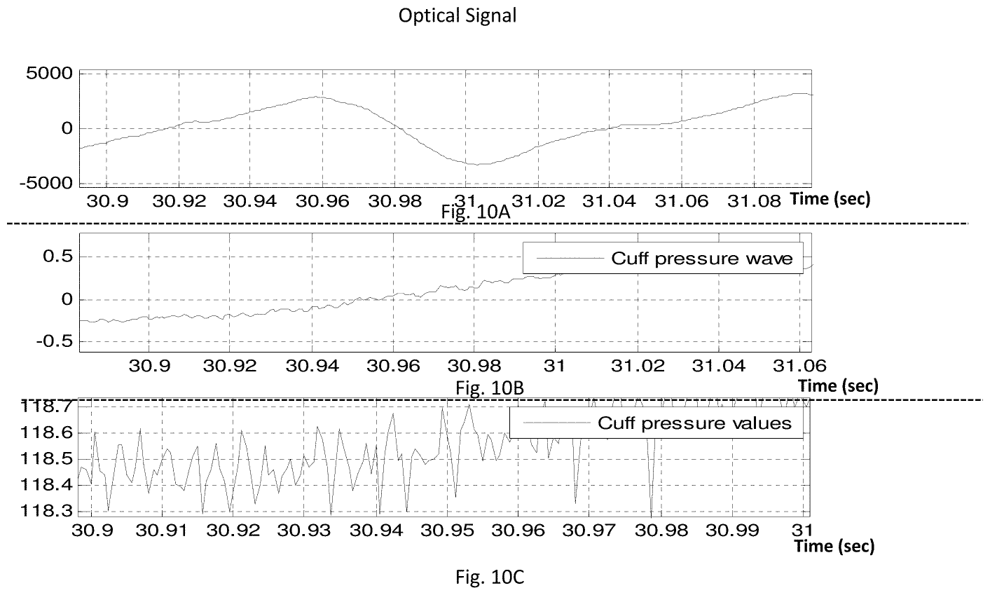

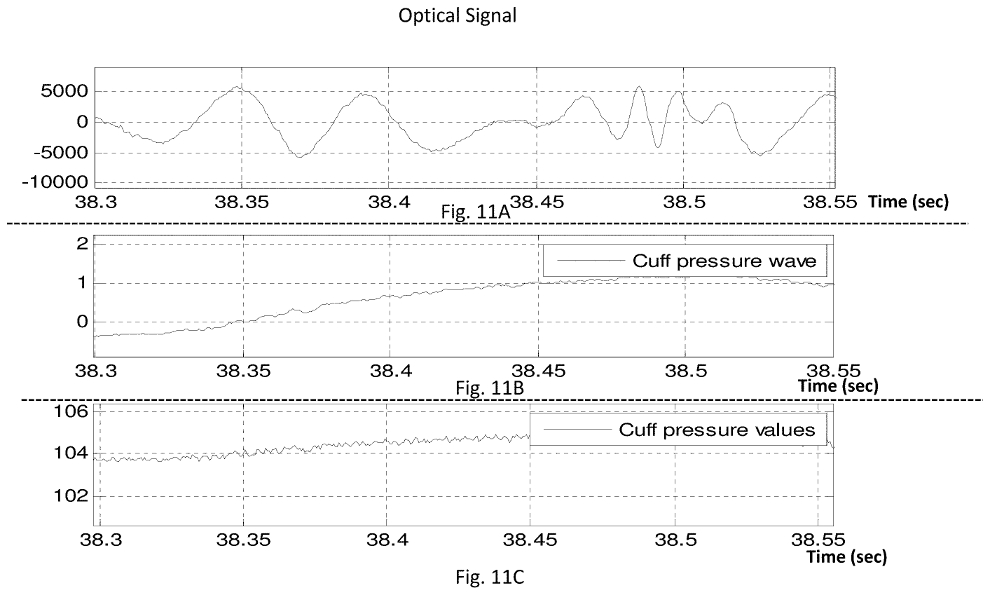

FIGS. 9A-9C relate to a situation before a first appearance of K-sound. FIGS. 10 and 11 show a first appearance of a K-sound. This first appearance corresponds to a systolic blood pressure of about 105 m Hg.

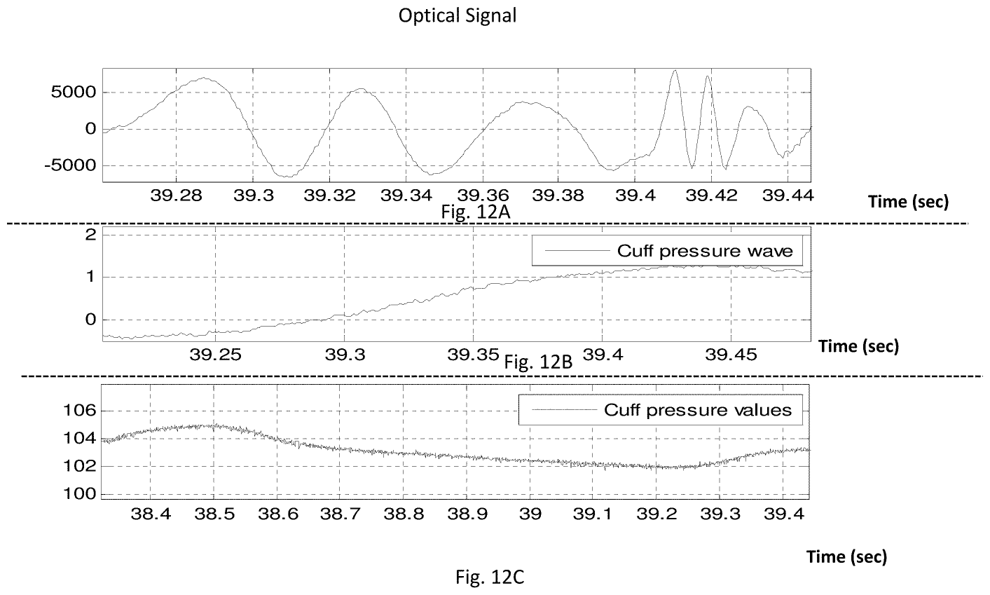

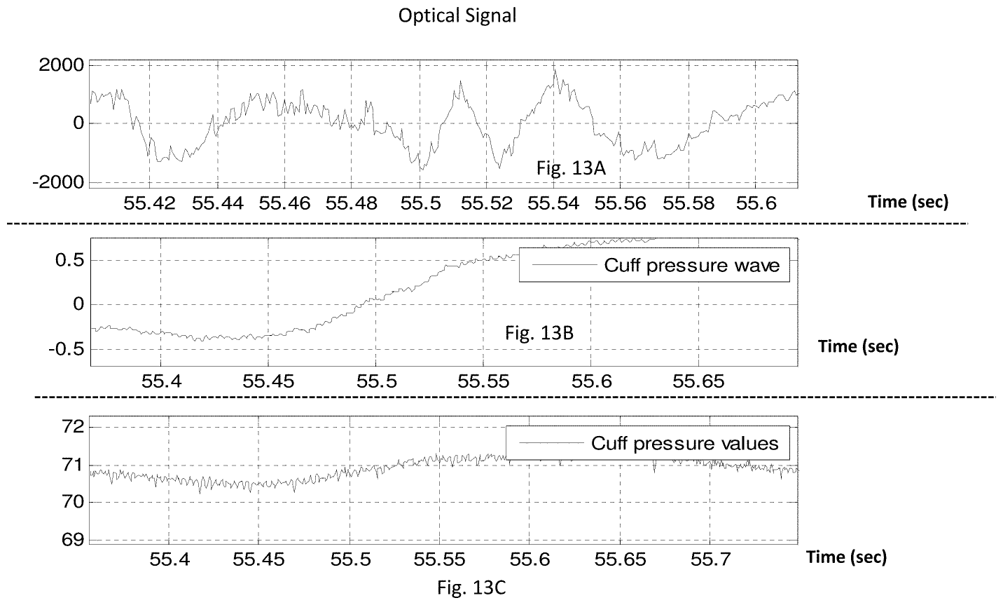

As shown in FIG. 12, the next/subsequent K-sound looks similar to the previous one. FIG. 13 shows the last appearance of K-sounds before its disappearance—this corresponds to the diastolic blood pressure. In FIG. 13 the diastolic point is 71 mm Hg.

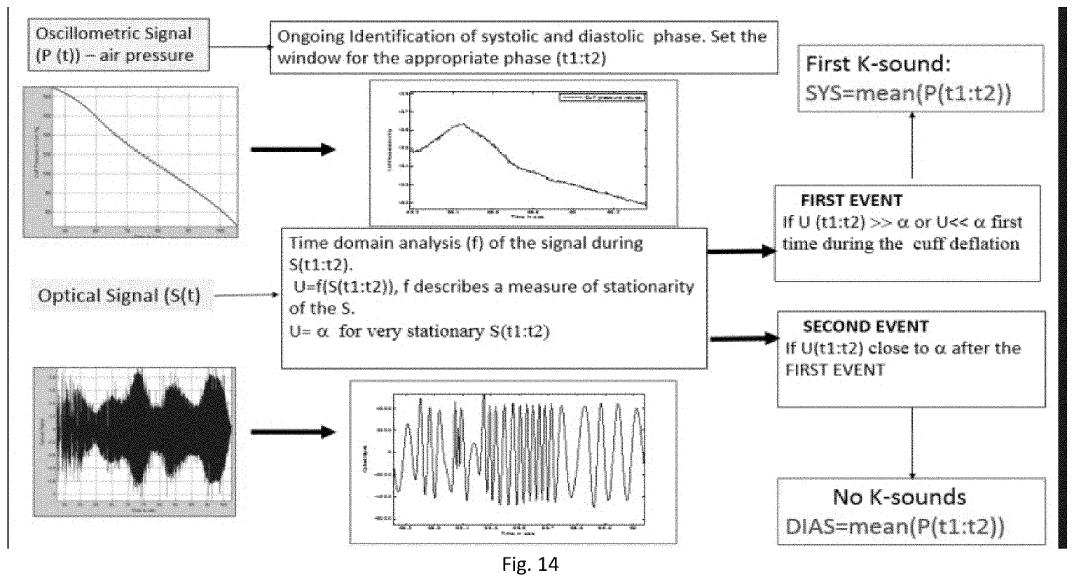

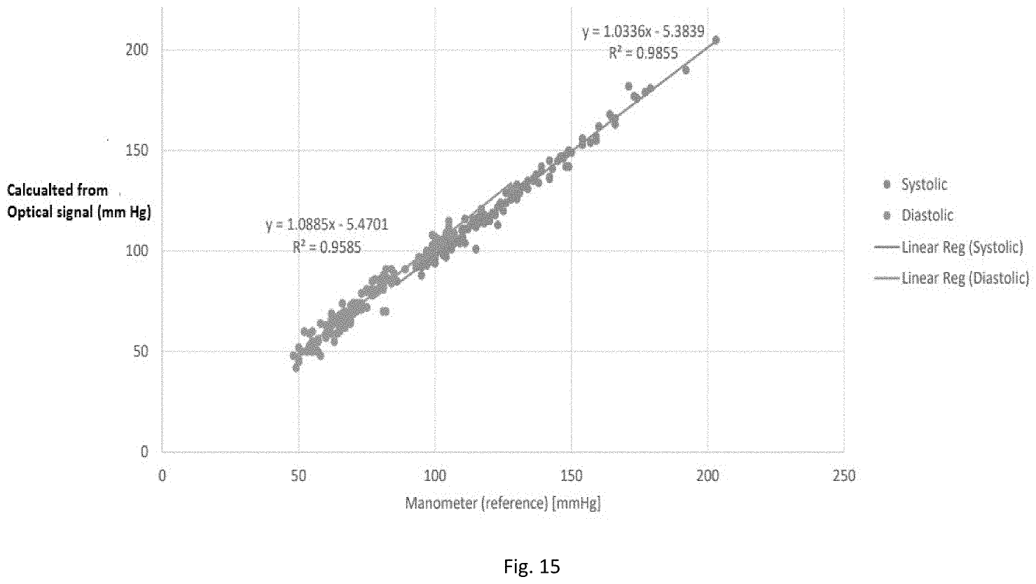

FIG. 14 shows a block diagram of an example algorithm for identifying K-sounds. FIG. 15 shows that blood pressure results obtained using techniques disclosed herein show a good correlations with results obtained using manual Ausculatory method (“Gold Standard BP results”).

Example Algorithm for Detecting K-Sounds

The algorithm exploits the Oscillometric pressure p(t) 370 and the optical signal 290 y(t). The Oscillometric pressure p(t) senses the heart pulsation while the optical signal senses as the heart pulsation so the K-sound.

K-sounds appears and, after a certain period, disappears during the release of the cuff pressure. The pressure when the K-sound appears is accepted as the systole, while the pressure when the K-sound disappears is accepted as the diastole.

The goal of the algorithm is to determine the systole and diastole by processing the sampled signals yn and signals pn.

Algorithm 1 comprises the following basic steps.

1. De-trending and pre-filtering of p(t) by the 0.5-5 Hz band-pass filter in order to extract the cardiac pulse waveform.

2. Detecting local maxima of p(t) and setting time bounds of each cardiac pulse.

3. Pre-filtering of y(t) by the 70-400 Hz band-pass filter in order to extract the K-sound.

4. Computing the power envelope of y(t).

5. Detecting the local maximum for each period of the cardiac pulse.

6. Detecting the standard deviation (invariant to the local maxima) for each period of the cardiac pulse.

7. Compute signal-to-noise ratio (SNR) as the ratio between the corresponding local maximum and standard deviation for each cardiac pulse period.

8. Detecting the first SNRs exceeding the accepted Systole Bound. The Systole Bound can be defined empirically using the k-sigma criterion, where k is number between 2-3 while “sigma” is the standard deviation of the three-five SNRs at the initial phase of the pressure deflation. The pressure related to this SNR will be interpreted as the systole.

9. Detecting the last SNRs exceeding the accepted Diastole Bound. The Diastole

Bound can be defined empirically using the k-sigma criterion, where k is number between 2-3 while “sigma” is the standard deviation of the three-five SNRs at the final phase of the pressure deflation. The pressure related to this SNR will be interpreted as the diastole.

There are some variants of the algorithm

Algorithm 2 is a modified version of Algorithm 1, where the power envelope is replaced by the Power Spectrum Density (PSD) as follows:

1. Compute the N-sample PSD for each segment of the de-trended acoustic signal yn related to the corresponding cardiac pulse period. In this case, the lower band power (from 0 to 70 Hz) serves as the noise power, while the higher band power (from 70 to 200 Hz) serves as the signal power. The ratio between the lower and higher band power of the PSD provides the desired SNR, which can be used further in steps 8 and 9 of the previous Algorithm.

Algorithm 3 is a modified version of Algorithm 2, in which the K-sound is tracked in the time-frequency domain in a manner accounting for the frequency shifts. The K-sound is indicated by an increase in the total power as well as in the noticeable shift toward higher frequency bands.

Theoretical Discussion

Laser Doppler frequency shift is a function of the velocity of the moving part of the membrane.

When the membrane 220 vibrates, the frequency of the reflected light is shifted. This shift is dependent on the velocity and the direction of the movement.

Each point on the membrane 220 reflects the light with a frequency multiplied by the factor given by well known expression:

$$Factor ( v ) = \frac{\sqrt{1 - {( \frac{v}{c} )}^{2}}}{1 + ( \frac{v}{c} ) * cos ( {⊖}_{0} )}$$

Where the frequency is shifted by : Shift=(1-Factor)

For V=v/c for small v we can approximate the Doppler shift by:

$$Df = {f}_{0} {(}_{1} - \frac{\sqrt{1 - {( \frac{v}{c} )}^{2}}}{1 + ( \frac{v}{c} ) * cos ( ⊖ )} ) ∼ {f}_{0} * V * cos ( ⊖ )$$

Where the central frequency f_0.

For example for wavelength 840 nm f_0 is about 3.5*10^14 Hz

θ represents the angle between the direction of the light propagation, and the observed direction of the light at reception point.

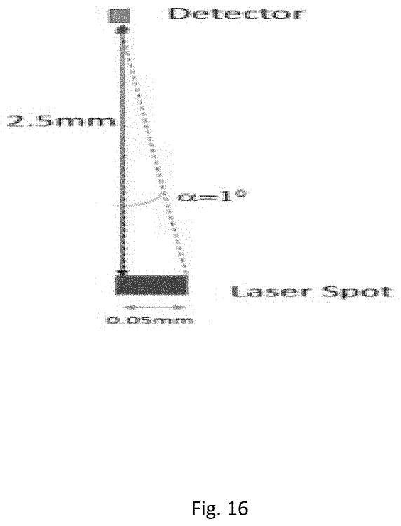

We can represent the light intensity amplitude at any given point of the detector, as a superposition of reflected waves with different frequencies, dependent on the angle between the spot location on the membrane surface and the detector. These waves come out from the illuminated spot from the light diffuser (FIG. 16).

Assuming that all points on the membrane moving with the same velocity V, the only optical path difference is predetermined by the angle of the light reflection. For one-dimensional case the overall measured signal amplitude is calculated as a summation of all waves. Each wave has different frequency shift. The superposition of all waves gives:

I=I₀∫π^{π-θ}sin(2πf₀(1+V*cos(θ))d(θ))

For cosine close to 1 we can use the following approximation:

cos(θ)˜−1+½(θ−π)²

Performing the integration we get:

I=Z1+Z2

### Where

$$Z 1 = \frac{1}{\sqrt{{f}_{0}} \sqrt{t} \sqrt{V}} ( - 0 .7 * Cos ( {f}_{0} t ( - 2 π + 2 π V ) ) * S ( \sqrt{f 0} \sqrt{t} ( - 4.4 + 1.4 * ⊖ ) \sqrt{V} ) \\ Z 2 = 0.7 C [ \sqrt{{f}_{0}} \sqrt{t} ( - 4.4 + 1.4 * ⊖ ) \sqrt{V} ] * Sin [ {f}_{0} t ( - 2 π + 2 π V ) ] )$$

### C and S are Fresnel's Integrals

S(x)=∫₀^{x}sin(t²)dt, C(x)=∫₀^{x}cos(t²)dt.

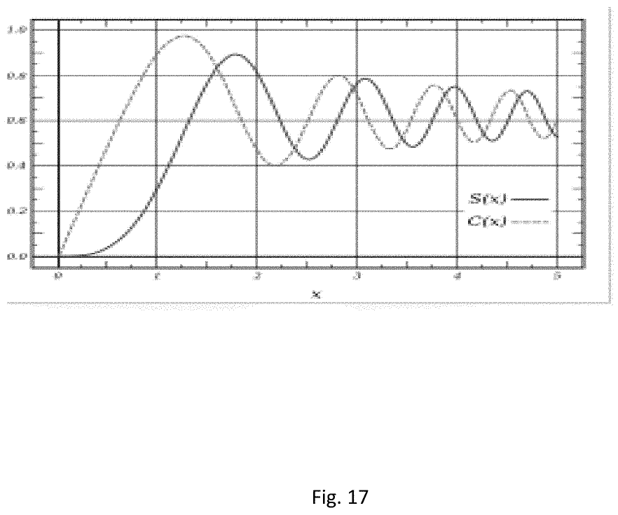

### Shown in FIG. 17.

Integration of I for v=1 mic/sec, and θ=0.05/2.5 reveals two components of the signal :

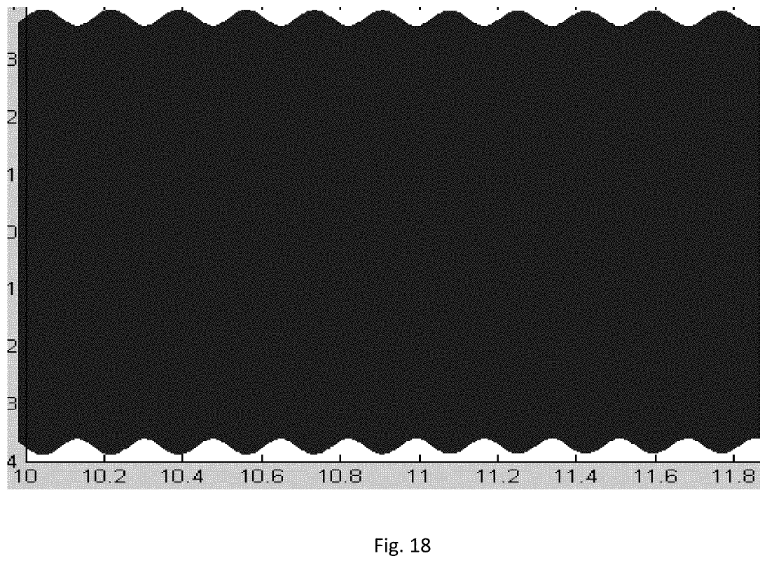

The high frequency component and low frequency component (see FIG. 18).

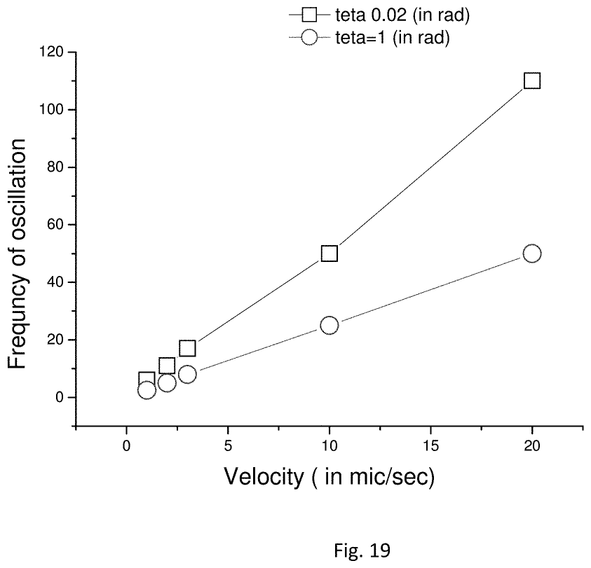

Since the high frequency component is averaged by low pass filter of the detection system the “demodulated signal” is measured. The frequency of its modulation dependent on the velocity and θ. FIG. 19 exemplifies this dependence.

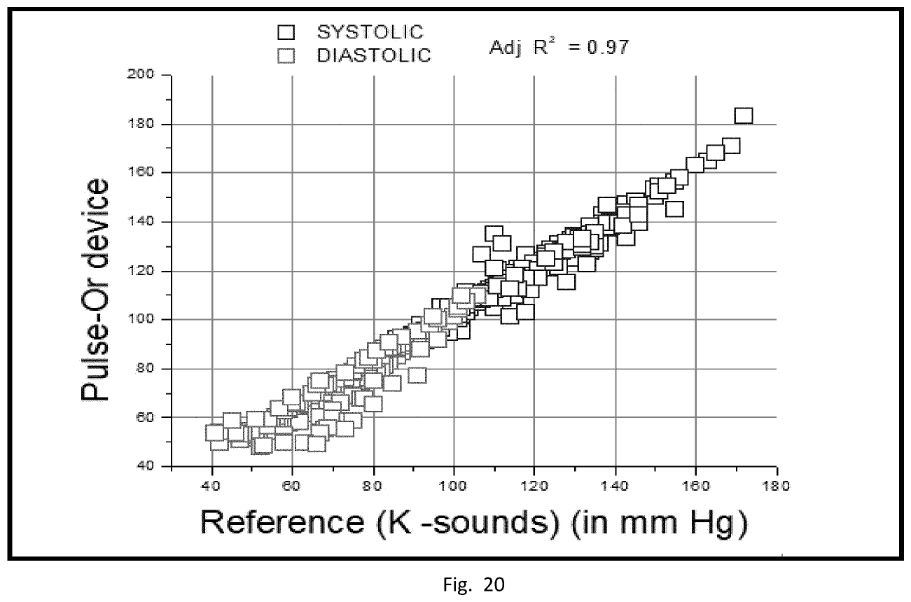

A discussion of FIGS. 20-23

FIG. 20 shows a correlation between a pulse-or device (e.g. used at the forearm) built according to presently disclosed teachings and a reference (e.g. gold standard) BP measurement.

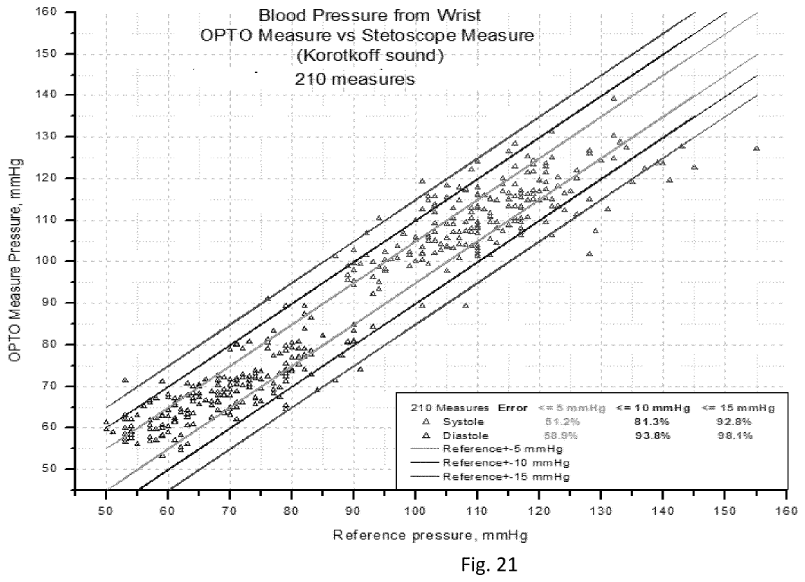

FIG. 21 shows a correlation between optical stethoscope device (e.g. used at the wrist) built according to presently disclosed teachings and a reference (e.g. gold standard) BP measurement.

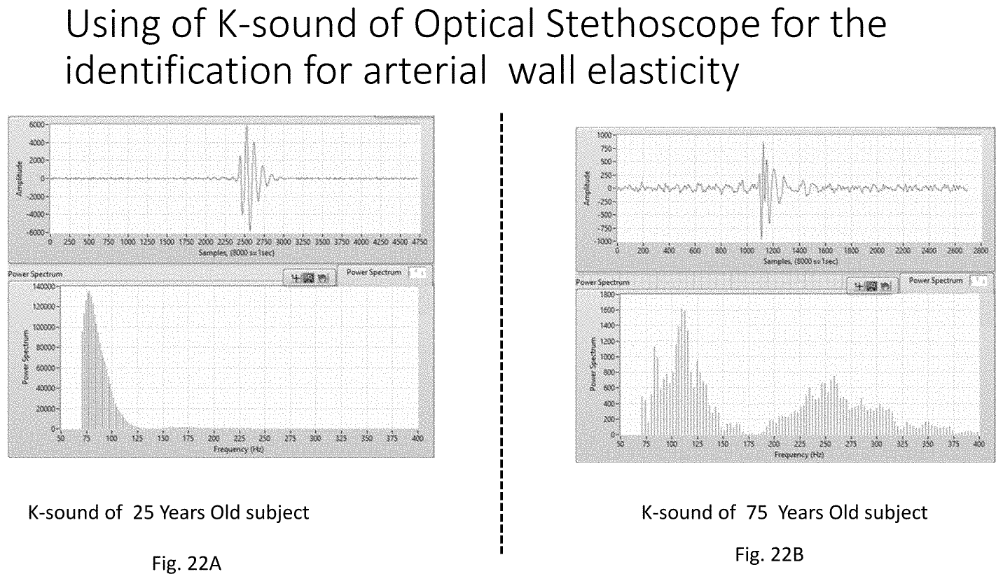

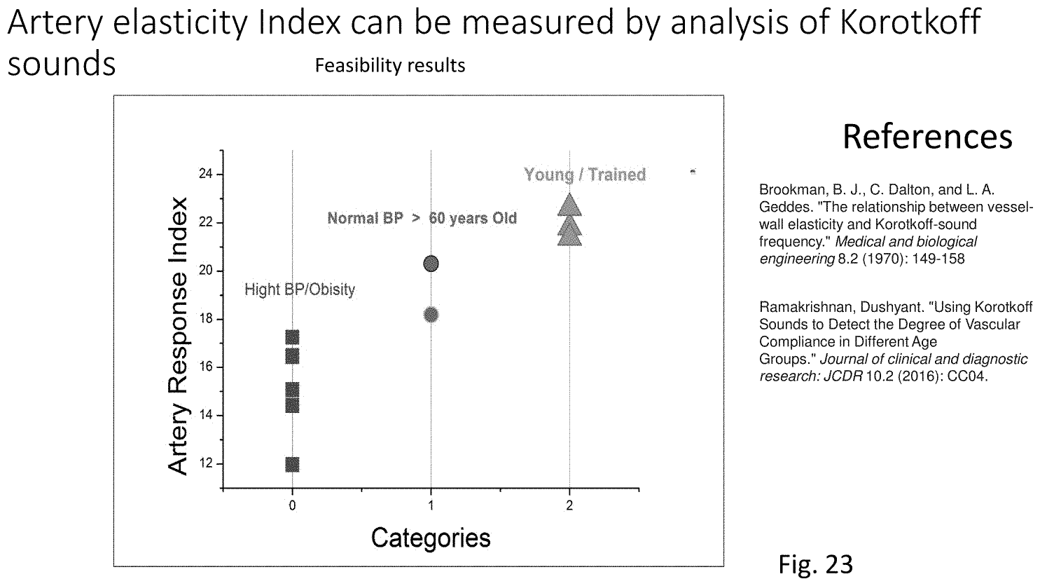

FIGS. 22A-22B and 23 relate to classifying different types of K sounds to identify arterial wall elasticity.

Additional Discussion

Concept 1: A system for noninvasive, real time method for identification of the Korotkoff sounds, comprising:
a blood pressure cuff;
at least one laser light source and at least one photodetector located in close vicinity to the laser light source where the laser beam is directed to the flexible membrane.
a flexible membrane pressed to the skin surface and capable to respond to the skin vibration related to the vibration of blood artery.
a sensor for the measurement of the pressure inside the air cuff
a controllable pump for inflating and deflating air cuff;
a processor for controlling the inflation and deflection of an air cuff
a computer implemented method for the identification of the Korotkoff sounds based on the analysis of the time dependent optical signal of the laser light source as it reflected by the membrane and is originated by the membrane vibration.

2. The system of concept 1 wherein a computer implemented method identifies the first and last Korotkoff sounds during deflation of cuff.

3. The system of concept 2 wherein a computer implemented method identifies the first Korotkoff sounds during inflation of cuff.

4. The system of concept 1 wherein a computer implemented method for the identification of the Korotkoff sounds is based on optical signal pattern recognition and analysis of irregularities of the time dependent measured optical signal during inflation or deflation of cuff.

5. The system of concept 1 wherein a computer implemented method for an identification of the Korotkoff sounds is based on spectrum analysis of the optical signal.

6. The system of concepts 1 and 2 wherein a computer implemented methods provides systolic and diastolic blood pressure according to the readings of the air cuff pressure at the first Korotkoff sound and the last Korotkoff sound.

7. The system of concept 1 where wherein the flexible membrane is covered by an optical diffuser.

8. The system of concept 1, wherein the cuff, probe and membrane are located at the upper arm of a subject.

9. The system of concept 1, wherein the cuff , probe and membrane are located at the wrist of a subject.

10. A method for noninvasive, real time measurement of the systolic and diastolic arterial blood pressure of a patient, comprising:
a) a blood pressure cuff and placing the cuff around a limb of the patient;
b) a sensor probe consisting of at least one coherent light source and at least one photodetector located in close vicinity to the coherent light source
c) a sensor probe located near a flexible membrane, so that the light emitted from a coherent light source if reflected from this flexible membrane where the other side of this flexible membrane is tightly pressed to a body part of a subject;
d) in putting the measured and photo signal into a processor, wherein the processor analysis an optical response of the body originated acoustic signal associated with the vessels response to the changes of the applied cuff pressure;
e) Inflating the cuff over systolic blood pressure
f) Gradually deflecting a cuff while the specially designed algorithm continuously process the time dependent characteristic pattern of the optical signal till identifies the prominent appearance of the characteristics of the Korotkoff sounds and the pressure measured in the air cuff at this specific moment is assigned to the systolic blood pressure
g) Further deflating the cuff time while the specially designed algorithm continuously process the time dependent characteristic pattern of the optical signal till identifies the disappearance of the Korotkoff sounds and the pressure measured in the air cuff at this specific moment is assigned to the diastolic blood pressure

11. The method of concept 10 wherein the specially designed algorithm is based on the analysis of irregularities of the optical signal fluctuation.

12. The method of concept 10 wherein the specially designed algorithm is based on the analysis of the correlation of the irregularities of the optical signal fluctuation with the oscillometric pulse wave.

1. Apparatus for optically measuring blood pressure and/or detecting Korotkoff-sounds of an animal, the apparatus comprising:
a. an inflatable cuff mechanically engageable to biological tissue of the animal;
b. a diffused-light interferometer optical stethoscope comprising:
i. a flexible and light-diffusing membrane;
ii. a coherent-light source configured to emit light having a visible or NIR wavelength λ, the coherent-light source being aimed at a surface of the flexible membrane; and
iii. a light-detector for receiving wavelength light λ that is emitted by the coherent-light source and reflected by the membrane,
the diffused-light interferometer optical stethoscope being configured such that the wavelength light λ received by the light-detector is primarily light that is diffuse-reflected by the membrane; and
c. Korotkoff-sound analysis circuitry for processing output of the light-detector that is generated when the flexible membrane is disposed over and/or mechanically engaged to and/or in contact with the cuff-engaged biological tissue, the Korotkoff-sound analysis-circuitry configured to detect Korotkoff-sounds from output of the light-detector.

2. Apparatus of claim 1 configured to optically measuring blood pressure and/or detecting Korotkoff-sounds of a human.

3. Apparatus of claim 1 wherein the light-diffusing membrane is a multi-layer assembly comprising a light-diffusing film disposed over a membrane that is optionally light-diffusing.

4. Apparatus of claim 1 wherein the light-diffusing membrane is substantially non-transparent to normally incident light of the wavelength λ so that optical density (OD) at wavelength λ is at least 2 or at least 3.

5. Apparatus of claim 1 wherein the diffused-light interferometer optical stethoscope is configured such that at least 80% or at least 90% or at least 95% (by power) of wavelength light λ received by the light-detector is primarily light that is diffuse-reflected by the membrane.

6. Apparatus of claim 1 wherein the coherent-light source is substantially normally aimed at a surface of the flexible membrane the coherent-light source being aimed at a surface of the flexible membrane, within a tolerance of at most 30° or within a tolerance of at most 20° or a tolerance of at most 15° or a tolerance of at most 10°.

7. Apparatus of claim 1 wherein (i) the coherent-light source produces a light-spot on the flexible membrane; (ii) the flexible membrane is held substantially flat to define a horizon; and (iii) a line-segment connecting a center to the spot of light to a center of detector is at least one of: (i) between 45° and 65°; (ii) between 40° and 70°; (iii) between 35° and 75°) above the horizon defined by substantially flat membrane.

8. (canceled)

9. Apparatus of claim 1 wherein the Korotkoff-sound analysis circuitry detects the Korotkoff sounds based on analysis of temporal irregularities of output of the light-detector.

10. Apparatus of claim 1 wherein further comprising blood-pressure detection circuitry for determining a systolic and/or diastolic blood pressure in accordance with a temporal correction between the Korotkoff-sound events and a pressure within the cuff.

11. Apparatus of claim 1 further comprising a pressure sensor for measuring a pressure within the inflatable cuff to generate an Oscillometric signal, and wherein the Korotkoff-sound analysis circuitry detects the Korotkoff events in accordance with a temporal correlation between (i) a pulsatile component of the Oscillometric signal and (ii) the output of the light-detector.

12. Apparatus of claim 1 wherein the Korotkoff-sound analysis circuitry distinguishes between Korotkoff sounds and other biological acoustic signals.

13. Apparatus of claim 1 wherein the Korotkoff-sound analysis circuitry detects the Korotkoff sounds by subjecting the optical signal to at least one of the following analysis techniques: entropy analysis, multiscale entropy analysis, fractal dimensions, multifractal analysis, wavelet analysis, Hurst exponential constants, pointwise Holder Exponent, and autocorrelation analysis.

14. Apparatus for optically detecting biological acoustic signals of an animal, the apparatus comprising:
a. an inflatable cuff mechanically engageable to biological tissue of the animal;
b. a diffused-light interferometer optical stethoscope comprising:
i. a flexible and light-diffusing membrane;
ii. a coherent-light source configured to emit light having a visible or NIR wavelength λ, the coherent-light source being aimed at a surface of the flexible membrane; and
iii. a light-detector for receiving wavelength light λ that is emitted by the coherent-light source and reflected by the membrane,
the diffused-light interferometer optical stethoscope being configured such that the wavelength light λ received by the light-detector is primarily light that is diffuse-reflected by the membrane; and
c. biological-acoustic-signal analysis circuitry for processing output of the light-detector that is generated when the flexible membrane is disposed over and/or mechanically engaged to and/or in contact with the cuff-engaged biological tissue, the biological-acoustic-signal analysis configured to detect biological acoustic signals of the animal from output of the light-detector.

15. (canceled)

16. The apparatus of any of claim 14 wherein the detected biological acoustic signals is selected from the group consisting of: (I) Korotkoff-sounds; (ii) a pulsatile acoustic signals; (iii) breathing or a pulmonary acoustic signal; (iv) a digestive or bowel acoustic signal; (v) an acoustic signal produced by a fetus within the animal; and (vi) sounds made by the heart, lungs, intestines, blood vessels vibration and/or blood flow.

17. Apparatus of claim 14 wherein the light-diffusing membrane is a multi-layer assembly comprising a light-diffusing film disposed over a membrane that is optionally light-diffusing.

18. Apparatus of claim 14 wherein the light-diffusing membrane is substantially non-transparent to normally incident light of the wavelength λ so that optical density (OD) at wavelength λ is at least 2 or at least 3.

19. Apparatus of any of claim 14 wherein the diffused-light interferometer optical stethoscope is configured such that at least 80% or at least 90% or at least 95% (by power) of wavelength light λ received by the light-detector is primarily light that is diffuse-reflected by the membrane.

20. Apparatus of any of claim 14 wherein the coherent-light source is substantially normally aimed at a surface of the flexible membrane the coherent-light source being aimed at a surface of the flexible membrane, within a tolerance of at most 30° or within a tolerance of at most 20° or a tolerance of at most 15° or a tolerance of at most 10°.

21. Apparatus of any of claim 14 wherein (i) the coherent-light source produces a light-spot on the flexible membrane; (ii) the flexible membrane is held substantially flat to define a horizon; and (iii) a line-segment connecting a center to the spot of light to a center of detector is at least one of: (i) between 45° and 65°; (ii) between 40° and 70°; (iii) between 35° and 75°) above the horizon defined by substantially flat membrane.

22-24. (canceled)

25. A method for optically measuring blood pressure and/or detecting Korotkoff-sounds of an animal, the apparatus comprising:
a. engaging an inflatable cuff to biological tissue of the animal;
b. providing a diffused-light interferometer optical stethoscope comprising:
i. a flexible and light-diffusing membrane;
ii. a coherent-light source configured to emit light having a visible or NIR wavelength λ, the coherent-light source being aimed at a surface of the flexible membrane; and
iii. a light-detector for receiving wavelength light λ that is emitted by the coherent-light source and reflected by the membrane,
the diffused-light interferometer optical stethoscope being configured such that the wavelength light λ received by the light-detector is primarily light that is diffuse-reflected by the membrane;
c. mechanically coupling the flexible membrane of the optical stethoscope to the biological tissue of the animal so that mechanical vibrations of a biological acoustic signal are conveyed from the biological tissue to the flexible membrane; and
d. electronically processing output of the light-detector that is generated when the flexible membrane is disposed over and/or mechanically engaged to and/or in contact with the cuff-engaged biological tissue so as to electronically detect Korotkoff-sounds from output of the light-detector.

26-27. (canceled)
